(19) United States
(12) Patent Application Publication
(10) Pub. No.: US 2014/0316007 A1
(43) Pub. Date: Oct. 23, 2014

(54) THERAPEUTIC COMBINATION OF MEMANTINE AND BACLOFEN AND PHARMACEUTICAL COMPOSITION CONTAINING THEM

(71) Applicant: RICHTER GEDEON NYRT., Budapest (HU)

(72) Inventors: Péter Kovács, Budapest (HU); Tamás Kitka, Budapest (HU); Melinda Misnyovszki, Budapest (HU); Balázs Varga, Budapest (HU); Sándor Farkas, Budapest (HU); Csilla Mária Horváth, Budapest (HU)

(73) Assignee: RICHTER GEDEON NYRT., Budapest (HU)

(21) Appl. No.: 14/356,096

(22) PCT Filed: Nov. 6, 2012
(86) PCT No.: PCT/HU2012/000119

§ 371 Date: May 2, 2014

(30) Foreign Application Priority Data
Nov. 7, 2011 (HU) P1100615

(51) Int. Cl. A61K 31/197; A61K 31/13

(52) U.S. Cl. CPC A61K 31/197; A61K 31/13
USPC 514/567

(57) ABSTRACT

The present invention relates to the combination of memantine and baclofen active ingredients, and also to the method for achieving body weight loss and thereby treating obesity and related co-morbidities by co-administration of baclofen and memantine.

### FIELD OF THE INVENTION

The present invention relates to the combination of memantine and baclofen active ingredients, wherein baclofen may also mean racemic baclofen, enantiomers and/or prodrugs of baclofen. The invention also relates to the use of such combinations in methods for treating overweight, obesity or related conditions or for achieving body weight loss, wherein memantine and baclofen are administered simultaneously or subsequently, preferably within a short period of time. The invention further relates to the pharmaceutical compositions comprising memantine and baclofen active ingredients and the use of such compositions in methods for treating overweight, obesity or related conditions or for achieving body weight loss. The treatment methods of the presented invention also mean a treatment regimen that is supplemented with other means such as dietary or life style modifications, dietary supplements, herbal or pharmaceutical remedies.

### BACKGROUND OF THE INVENTION

Overweight and obesity are growing public health problems in the modern world. In medical practice overweight is defined as a body mass index (BMI) above 25 kg/m2, while obesity as BMI>30 kg/m2. As of 2011, the prevalence of obesity more than doubled compared to 1980 (WHO Fact sheet N°311, 2011); http://www.who.int/mediacentre/factsheets/fs311/en/). In most parts of the world the prevalence of overweight and obesity grows progressively (Europe, USA, Middle East and Asia). According to the estimation of World Health Organisation (WHO), in 2008, the worldwide prevalence of overweight and obesity were 1.5 billion and 500 million, respectively. Both overweight and obesity, namely the increased body mass beyond healthy limits, increases the risk of several diseases in a severity dependent manner. In this description, hereafter obesity stands for both overweight and obesity categories. Obesity is a known risk factor for several diseases and medical conditions, such as diabetes, insulin resistance, metabolic syndrome, hypertension, atherosclerosis, coronary artery disease, cardiac failure, stroke, biliary tract diseases, such as cholecystitis and gallstones, osteoarthritis, orthopedic abnormalities, dyspnea, respiratory apnea, ovarian cysts, malignancies, such as mammary, prostate and colon tumors, anesthesiological complications, heartburn, venous varicosities, infections and eczema (Kopelman Nature 404:635-643 2000; Rissanen et al. BMJ 301:835-837 1990). Obesity also has a negative effect on life expectancy, and along with smoking, hypertension and hypercholesterolemia, it is one of the major risk factors for several chronic diseases (James, Comparative Quantification of Health Risks Global and Regional Burden of Diseases Attributable to Selected Major Risk Factors, Chapter 8, WHO, Geneva, 2004).

Overweight and obesity is the fifth leading risk factor for mortality worldwide. At least 2.8 million adults die yearly as a consequence of being overweight or obese (WHO Fact sheet N°311, 2011). The medical need for weight loss in obese people is underlined by the fact that as small as 5% long term weight loss is able to significantly improve cardiovascular morbidity and mortality rates (Goldstein, Int. J. Obes. Relat. Metab. Disord. 16:397-415 1992). Therefore, an enormous unmet medical need exists for the treatment of obesity and related comorbid conditions.

It is known that obesity can be alleviated with rigorous low calorie dieting and exercise. However, according to medical experience, these kind of lifestyle modifications are not effective on the long term and have limited utility in some patient populations (Powell et al. Am. Psychol. 62:234-246 2007; Sahoo, Obesity Drug Markets in the US and EU: Analysis of product pipelines and the competitive environment. Business Insights Ltd., 2008). Therefore, a large public demand exists for pharmacotherapies that can support hypocaloric dieting and can enhance the effectiveness of behavioral modifications (Witkamp Pharm. Res. 28:1792-1818 2011).

The progress of obesity is multifactorial, but at the end it always manifests as impairment in the regulation of energy intake and expenditure. Although the human body aims to maintain its weight, a moderate weight gain along with aging can be considered as a normal physiological process. However, in modern societies several environmental factors, such as sedentary lifestyle and the easy availability of energy-dense foods, severely affect the normal homeostatic control of body weight, which lead to increased storage of body fat (Bessesen Physiol. Behav. 104:599-607 2011). Accordingly, anti-obesity pharmacotherapies are aiming to reduce unutilized energy by (1) reducing energy absorption, (2) alleviating hunger/increasing satiety, thereby reducing energy-intake, or (3) increasing the utilization of stored energy (Witkamp Pharm. Res. 28:1792-1818 2011). Out of these three pharmacotherapeutic options the latter two can be achieved by using drugs acting in the central nervous system (CNS).

Some regions of the CNS play crucial role in the regulation of energy intake, energy expenditure and metabolism. For example, one main integrating center is the hypothalamus (Gao et al. Annu. Rev. Neurosci. 30:367-398 2007). More than 50 neurotransmitters have been identified in the hypothalamus that play a proven or potential role in the regulation of energy homeostasis. The neurotransmitters that are involved in the regulation of energy homeostasis provide potential targets for anti-obesity pharmacotherapies. However, since the development of obesity and the regulation of food intake and energy homeostasis is affected by various central and peripheral pathways—some of which are prone to fast adaptation and resistance—targeting only one pathway can hardly provide an effective pharmacotherapy (Aronne et al. Expert Opin. Emerg. Drugs 16:587-596 2011). Accordingly, targeting more pathways—e.g. by co-administering simultaneously more than one drug—might be needed to reach an optimal therapeutic effect. Nevertheless unpredictable interactions might occur when using combination therapies, especially if CNS mechanisms are involved. These interactions can range from antagonistic interaction through additive effect to supra-additive interaction (synergy).

Reaching sufficient efficacy is a fundamental requirement with regards to therapeutic utility. Namely, if the efficacy of an anti-obesity pharmacotherapy does not reach a certain limit, drug licensing agencies (Food and Drug Administration, FDA in the USA or European Medicines Agency, EMA in Europe) would not grant the approval of the drug. According to the actual FDA guidelines, the required mean primary efficacy endpoint is 5% weight loss (versus placebo-treated group) at 1 year, while the categorical primary efficacy endpoint requires that at least 35% of the treated population should lose more than 5% body weight (versus placebo-treated group) (Guidance for Industry Developing Products for Weight Management. U.S. Department of Health and Human Services, FDA (2007)). The desirable minimum weight loss specified by EMA is even higher: 10% (Guideline On Clinical Evaluation Of Medicinal Products Used In Weight Control. EMA (2008)).

However, it is not enough for an anti-obesity drug to be sufficiently effective. In addition, drug regulatory agencies set very high safety standards for these kinds of drugs. Therefore such drugs must be substantially devoid of side effects at therapeutic doses. Despite that efficient anti-obesity therapy may require centrally acting drugs, at the time of writing this application there are no approved drugs on the market which are indicated for long term use. This lack of CNS drugs is at least partly due to the high safety and tolerability standards. The only approved anti-obesity treatment for long term use is a non-CNS drug, orlistat, which blocks digestion and subsequent absorption of alimentary fat. However, efficacy of orlistat is fairly less than desirable and its broad use is also limited by troublesome gastrointestinal side effects (Filippatos et al. Drug Saf. 31:53-65 2008). Achieving a sufficient therapeutic index, i.e. good separation of effective and side effect causing doses, is a particularly challenging task with CNS drugs. Two CNS drugs, rimonabant and sibutramine were recently withdrawn from the market due to unwanted side effects (Kennett et al. Pharmacol. Biochem. Behav. 97:63-83 2010). Consequently, achieving appropriate efficacy and sufficient lack of side effects, hence good tolerability and safety are critical and challenging issues in the pharmacotherapy of obesity. In another aspect, suitable separation between doses mediating efficacy and side effects is also an essential criterion in order to meet the requirements of good tolerability and safety.

Severely obese patients (BMI>35-40) with comorbid conditions (e.g. diabetes, hypertension) who are unresponsive to diet and pharmacotherapy are treated in some countries by gastrointestinal surgical interventions, called bariatric surgery (Powell et al. Am. Psychol. 62:234-246 2007). However, such surgical interventions have considerable risks, including mortality, severe postoperative side effects and high rate of postoperative complications (Encinosa et al. Med. Care 44:706-712 2006). Despite the substantial risks, the severely obese population can still benefit from surgical treatments, due to the high impact of obesity-related comorbidities on life expectancy and on the quality of life. Therefore, it is a reasonable assumption that a future pharmacotherapy providing high efficacy close to that of the surgery, i.e. 20-25% body weight loss (Bueter et al. Obes. Facts 2:325-331 2009) along with less risks and side effects, could offer a better treatment option for severely obese patients.

Recently, it turned out, that several drugs—that were originally developed to treat other diseases—have some body weight reducing effects in humans at their regular therapeutic doses (e.g. zonisamide, topiramate, bupropion, naltrexone (Kennett et al. Pharmacol. Biochem. Behav. 97:63-83 2010); atomoxetine (Gadde et al. Int. J. Obes. (Lond) 30:1138-1142 2006); baclofen (Arima et al. Intern. Med. 49:2043-2047 2010); betahistine (Barak et al. Int. J. Obes. (Lond) 32:1559-1565 2008); duloxetine (Guerdjikova et al. In.t J. Eat. Disord. Epub ahead of print 2011); fluoxetine (Serretti et al. J. Clin. Psychiatry 71:1259-1272 2010); memantine (Hermanussen et al. Econ. Hum. Biol. 3:329-337 2005); methylphenidate (Leddy et al. Obes. Res. 12:224-232 2004); sertraline (Serretti et al. J. Clin. Psychiatry 71:1259-1272 2010); venlafaxine (Malhotra et al. J. Clin. Psychiatry. 63:802-806 2002). On the other hand, these medications usually have modest efficacy, typically below 5% body weight loss compared to placebo or baseline. Furthermore, it is also questionable whether their side effect profile would be acceptable in view of the high regulatory safety bars in the obesity indication.

It has also been known for quite a long time that amphetamines and similar drugs (e.g. phentermine, diethylpropion, phendimetrazine, phenylpropanolamine, mazindol) have anti-obesity effects. Nonetheless, the majority of these kinds of medications had been withdrawn from the market, due to cardiovascular risks, abuse potential and psychostimulant side effects (Ioannides-Demos et al. Drug Saf. 29:277-302 2006). Their therapeutic utility is also limited by their liability to development of tolerance, which leads to attenuation or cessation of efficacy over time during long-term treatment. Therefore, marketing authorization of most of these compounds has been withdrawn and the few commercially still available amphetamine-like compounds, like phentermine in the USA, are only permitted for short term treatment (Kennett et al. Pharmacol. Biochem. Behav. 97:63-83 2010).

In summary, several drugs exist with weak anti-obesity activity, which are either insufficiently effective or carry unacceptable side effect profiles when administered alone. However, it is absolutely not obvious that combinations of which drugs result in real summation of the effects (additive interaction) and which combinations do not lead to such summation (infra-additive interaction). Furthermore, it is even less obvious which drug combinations exhibit an even higher efficacy than would be expected from simple summation (i.e. supra-additive interaction or synergy) in terms of the desired action. It is also not obvious whether the components of the combinations will enhance or reduce each-others' side effects or ultimately the combination will have better or worse side effect profile and therapeutic index than the components alone. In the case of synergistic action for the desired action it is also questionable whether or not the synergy refers to the side effects as well. As a matter of fact, synergy for both main and side effects would not yield an improved therapeutic index. Hence, it is not obvious at all whether the combination of two drugs with known anti-obesity effects yields a combination that is favorable in terms of therapeutic utility and benefits as compared to solo use of its components.

Baclofen has been used for a long time as a centrally acting muscle relaxant drug. Its primary pharmacological action is an agonist effect on the gamma-aminobutyric acid B-type (GABA-B) receptors (Davidoff, Ann. Neurol. 17:107-116 1985). The drug used in medical practice is a racemic mixture of left-(S-) and right-handed (R-) enantiomers. Its oral form is used to alleviate spasticity associated with CNS injury related disorders which cause an increase in muscle-tone that is called ‘spasticity’. Its most common side-effect is muscle weakness due to exaggerated muscle relaxation, for which its therapeutic index is rather narrow. In addition, drowsiness and dizziness are also common side effects of baclofen. Therefore, for attaining an efficacious anti-spastic but well tolerable dose level individual dose titration is recommended. The effective therapeutic doses of oral baclofen for the treatment of spasticity fall typically in the range of 30-80 mg/day (Dario et al. Drug Saf. 27:799-818 2004). Similarly to humans, the anti-spastic and motor side effect-causing doses do also overlap in mice (Farkas et al. J. Pharmacol. Toxicol. Methods 52:264-273 2005).

It is known that baclofen decreases food intake and body weight in diet induced obese mice. Both enatiomers of baclofen had body weight reducing effects, however the R-enantiomer was more effective (Sato et al. FEBS Lett. 581:4857-4864 2007). The moderate effect of baclofen on body weight has also been proven in a small human study (10 obese patients), in which baclofen was administered at the dose of 30 mg/day, beginning with a 10 day gradual dose-increasing phase and lasting for 12 weeks. The study showed a mean 1.7% body weight loss. Out of the 10 participants only one had lost more than 5% body weight (Arima et al. Intern. Med. 49:2043-2047 2010). These limited data suggest that baclofen treatment alone at well tolerated doses would not meet the minimum efficacy criteria of drug regulatory agencies. It is known, for example, that a new drug candidate lorcaserin, which was entitled by the FDA (in its “complete response letter” of 2010) as having “marginal efficacy”, caused 3.5-4% weight loss at 12 weeks and 5.8% after 1-year-long treatment relative to baseline. Hence, the less than 2% body-weight decreasing effect of 30 mg/day baclofen in 12 weeks can be considered as submarginal. There is another human baclofen study that refers to food intake reducing effect of baclofen, though that study was not designed to assess anti-obesity efficacy. Broft and her co-workers (Broft et al. Int J. Eat. Disord. 40:687-691 2007) investigated the effects of baclofen on binge eating in seven female participants. Binge eating disorder is not equivalent with obesity and out of the 7 patients only 2 were obese (BMI>30 kg/m²) and 1 was overweight (BMI>25 kg/m²). In this investigation there were no significant changes in mean body weight (0.9 kg body weight increase within 10 weeks) but only food craving and the number of binge eating episodes were decreased by baclofen. The targeted dose of baclofen was 60 mg/day and the most common side effect was sedation. Hence, these data indicate that baclofen has some moderate appetite reducing effects but this effect alone is not sufficient to cause a clinically meaningful body weight loss at doses associated with no side effects or at least with a tolerable side effect profile.

Memantine has been an approved drug for quite a long time. Initially (in 1978) it entered the market in Germany for the treatment of Parkinson's disease, spasticity and other neurological disorders. Later it was found that memantine blocks N-methyl-D-aspartate (NMDA) receptors in a noncompetitive manner (Bormann, Eur. J. Pharmacol. 166:591-592 1989), shows neuroprotective effects and is also effective in preventing cognitive and histological damages in preclinical models of Alzheimer's disease (Parsons et al. Neuropharmacology 38:735-767 1999); Rammes et al. Curr. Neuropharmacol. 6:55-78 2008). Then its efficacy was proven also in clinical trials of vascular dementia and Alzheimer's disease (Raina et al. Ann. Intern. Med. 148:379-397 2008). Currently memantine is an approved and widely used drug for the treatment of Alzheimer's disease. Its general therapeutic dose is 20 mg/day in the clinical practice, which should be reached only by gradual dose-escalation. Its therapeutic window is narrow and side effects typical for NMDA antagonists, such as restlessness, confusion, or more seriously hallucination, may occur in case of too fast dose-escalation or administration of higher doses. Nevertheless, side effects of memantine are rare when administered according to the recommended dosing regimen: restlessness (1.3%), nausea (0.9%), dizziness (0.8%), tiredness (0.4%) (Mobius et al. Drugs of Today 40:685-695 2004). It is difficult to determine which dose in the animal experiments corresponds to the human therapeutic dose. However, the effective anti-Alzheimer dose range of memantine in mice can be estimated to be 5-30 mg/kg/day with oral administration based on plasma concentration data or cognitive and neurohistological effects in animal models of Alzheimer's disease (Dong et al. Neuropsychopharmacology 33:3226-3236 2008); Minkeviciene et al. J. Pharmacol. Exp. Ther. 311:677-682 2004); Rammes et al. Curr. Neuropharmacol. 6:55-78 2008).

In an open-label clinical trial on 5 obese female patients (Hermanussen et al. Econ. Hum. Biol 3:329-337 2005), memantine, at doses higher than the usual therapeutic dose (20-30 mg/day with dose adjustment if needed) was found to decrease the appetite, number binge eating episodes and body weight. However, the relevance of this observation with regards to assessment of utility for anti-obesity treatment is very limited, since all but one of these patients were treated only for a short period (21 or fewer days). Some patients experienced dizziness during the treatment. In a more extended open-label study, where memantine was administered to 16 obese, binge eater patients according to the dosing regimen recommended in labeling (i.e. the dose was gradually increased to 20 mg/day or left lower if needed for good tolerability), memantine decreased the number of binge episodes but not the body weight (Brennan et al. Int. J. Eat. Disord. 41:520-526 2008). Hence, these data suggest that although a slight decrease in appetite can be reached using memantine but the well-tolerated doses in current therapeutic use do not cause a substantial weight loss. This conclusion is in accordance with data from animal experiments where memantine decreased binge numbers in a rat model of binge eating disorder but did not decrease body weight (Popik et al. Amino Acids 40:477-485 2011).

### SUMMARY OF THE INVENTION

This invention is based on the unexpected observation that combined application of memantine and baclofen exerted a surprisingly strong, apparently synergistic effect on weight loss in a mouse model of obesity. In contrast, combined administration of baclofen with phentermine, which is another drug with a known weight reducing effect, caused clearly an infra-additive interaction. Furthermore, as another unexpected finding, the synergistic and remarkable weight-reducing effects of memantine and baclofen were found at doses that were lower than the doses sufficient to detect their known therapeutic efficacy in relevant mouse models related to their current indications.

We have also surprisingly found that the combination of memantine and baclofen exhibited infra-additive interaction in terms of side effects and resulted in improvement of the therapeutic index.

The present invention relates to the combination of memantine and baclofen active ingredients, wherein baclofen may also mean racemic baclofen, enantiomers and/or prodrugs of baclofen.

The invention further relates to the pharmaceutical compositions comprising memantine and baclofen active ingredients.

The invention also relates to the use of combinations and compositions of memantine and baclofen active ingredients in methods for treating overweight, obesity or related conditions or for achieving body weight loss.

### BRIEF DESCRIPTION OF THE FIGURES

FIG. 1 depicts the efficacy of memantine, baclofen and their combination in the mouse DIO test.

FIG. 2 shows the magnitudes of the weight reducing effects of memantine, baclofen and their combination in the mouse DIO test (pooled results of two studies).

FIG. 3 shows the efficacy of phentermine, baclofen and their combination on the mouse diet-induced obesity test. The doses presented were administered per os twice daily.

FIG. 4. shows the isobolographic analysis of the pharmacological interaction between memantine and baclofen in the rotarod test in mice.

FIG. 5. shows the effect of memantine, baclofen and their combination on the horizontal motor activity of mice.

### DETAILED DESCRIPTION OF THE INVENTION

The present invention relates to the combination of memantine and baclofen active ingredients, wherein baclofen may also mean racemic baclofen, enantiomers and/or prodrugs of baclofen and to the use of such combinations in methods for treating overweight, obesity or related conditions or for achieving body weight loss, wherein memantine and baclofen are administered simultaneously or subsequently, preferably within a short period of time. The invention further relates to the pharmaceutical compositions comprising memantine and baclofen active ingredients and the use of such compositions in methods for treating overweight, obesity or related conditions or for achieving body weight loss. The treatment methods of the presented invention also mean a treatment regimen that is supplemented with other means such as dietary or life style modifications, dietary supplements, herbal or pharmaceutical remedies.

In course of our experiments, we have surprisingly found that the combination of memantine and baclofen cause a remarkable weight reduction even when applied at doses below their present human therapeutic dose. In addition, if a similar synergism is not found in their side effects, then the improvement of the side effect profile and therapeutic index can also be experienced by the combination of these drugs. We have proven that no synergy was present in terms of CNS side effects typical for baclofen, i.e. muscle weakness and dizziness, as measured by rotarod performance, when administered these drugs to mice at a dose combination that exerts an apparent synergistic effect in terms of weight reduction. Moreover, unexpectedly we observed that baclofen counteracted the locomotor activity-increasing effect of memantine, an effect that can be observed in mice after treatment with memantine and which is typical for NMDA antagonist compounds.

Based on the results of our experiments, the following key features of the new combination can be summarized:

(1) A weight reduction higher than the acceptable threshold value proposed by the FDA might be reached using the combination of memantine and baclofen.

(2) This efficacy can be reached at a dose of baclofen that is lower than its usual therapeutic dose or falls in the lower end of the recommended dose range according to current labeling (Summary of Product Characteristics). Accordingly the stipulated anti-obesity dose range for baclofen is 5-40 mg/day depending on the weight of the patient.

(3) In the case of memantine a successful weight reducing effect can be reached at its usual therapeutic dose (20 mg/day) or at lower doses (2-20 mg/day).

(4) Due to the lower doses and/or to counteracting effects of the components concerning side effects, a better therapeutic window and side effect profile can be observed with combination of memantine and baclofen.

(5) In cases of morbid obesity that carries higher health risks and may need very high efficacy, slightly inferior side effect profile is acceptable in the risk/benefit evaluation. Therefore in such cases the combination of these compounds can be applied at doses in the upper region of their usual therapeutic dose range. Moreover, in accordance with the clinical practice, there may be a need to adjust doses to higher body weights in the case of extremely heavy patients (>120 kg). Therefore application of higher doses than the above mentioned ones may be reasonable in certain cases, particularly in cases of morbid obesity (i.e. baclofen 20-160 mg, memantine 10-40 mg).

According to the present invention the combinations of memantine and baclofen active ingredients preferably contain memantine in the range of about 2 to about 40 mg/day and baclofen in the range of about 5 to about 160 mg/day. In a further preferred embodiment the combination contains memantine in the range of about 2 to about 20 mg/day and baclofen in the range of about 5 to about 40 mg/day. In case of serious need the combination may more preferably contains memantine in the range of about 10 to about 40 mg/day and baclofen in the range of about 20 to about 160 mg/day.

The invention also relates to the pharmaceutical compositions comprising memantine and baclofen combinations and pharmaceutically acceptable excipients.

Suitable routes of administration may, for example, include oral, rectal, transdermal administration or parenteral delivery. The pharmaceutical compositions of the invention can be formulated as liquids or solids, for example solutions, suspensions, emulsions, liposomes, granules, tablets, film-tablets or capsules.

The pharmaceutical compositions can be administered by variety of routes and dosage forms. The memantine and baclofen active ingredients can be formulated into a pharmaceutical composition either in combination or separately and the compositions can be administered in either single or multiple doses.

The dosage required to exert the therapeutic effect can vary within wide limits and will be fitted to the individual requirements in each of the particular case, depending on the stage of the disease, the condition and the bodyweight of the patient to be treated, as well as the sensitivity of the patient against the active ingredient, route of administration and number of daily treatments. The actual dose of the active ingredient to be used can safely be determined by the attending physician skilled in the art in the knowledge of the patient to be treated.

For the sake of a simple administration it is suitable if the pharmaceutical compositions comprise dosage units containing the amount of the active ingredient to be administered once, or a few multiples or a half, third or fourth part thereof. Such dosage units are e.g. tablets, which can be powdered with grooves promoting the halving or quartering of the tablet in order to exactly administer the required amount of the active ingredient.

The pharmaceutical compositions containing the active ingredients according to the present invention usually contain 3 to 200 mg of active ingredients meaning preferably 1 to 40 mg of memantine and 2 to 160 mg of baclofen in a single dosage unit. In a further preferred embodiment the composition contains 1 to 20 mg of memantine and 2 to 40 mg of baclofen in each dosage unit. Depending on the stage of the disease the compositions may more preferably contain 5 to 40 mg of memantine and 10 to 160 mg of baclofen in each dosage unit.

It is, of course possible that the amount of the active ingredient in some compositions or combinations exceeds the upper or lower limits defined above.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

The aim of the pharmaceutical formulation procedure of the present invention is to develop a new weight-loss promoting, oral pharmaceutical composition containing the two active ingredients and to elaborate a procedure for the reproducible industrial production of the product assuring homogenous distribution of the two active ingredients in the composition and warranting the stability of the composition till the end of the expiration date, satisfying all the strict pharmaceutical regulatory, stability and safety demands. Through the suitable industrial procedures the active ingredients are formulated into capsules, tablets, filmtablets, capsules filled with pellets or tablets, filmtablets derived from pellets.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art.

The active ingredients can be mixed with for example lactose, cellulose, starch, sucrose, mannitol, sorbitol, calcium phosphate and calcium sulphate as commonly used diluents. The microcrystalline cellulose functions not only as a diluent; it has also some lubricant and disintegrant properties that make it beneficial. Calcium carboxymethyl amylopectin, sodium carboxymethyl amylopectin, croscarmellose sodium, polyvinylpyrrolidone, starches can be added among others as disintegrants; gelatin, hydroxypropyl cellulose, hydroxypropyl methylcellulose and polyvidone can be used among others as binders; and other excipients can be added to modify the solubility and/or release of the active ingredients.

To the powder or granule mixture, if necessary, at any operational steps additional excipients e.g. colloidal silicon dioxide, talc, calcium stearate, glyceryl monostearate, magnesium stearate, polyethylene glycol, sodium stearyl fumarate, stearic acid and zinc stearate are added as lubricants or glidants and/or different colouring and/or flavouring agents and/or additives modifying the drug release can be used. The compressed tablets or filled capsules can be film or sugar-coated.

The above described ingredients and different routes of manufacture are merely representative. Other materials as well as processing techniques and the like well known in the art can also be used.

The combinations and compositions of the present invention are useful for achieving body weight loss and for the treatment of overweight, obesity or related conditions. Consequently the invention relates to the method of treatment of overweight and obesity in a mammal, particularly in human by administering an effective amount of memantine and baclofen. The combinations of memantine and baclofen are administered simultaneously or subsequently.

### Studies and Results Substantiating the Invention

### Studying the Efficacy on Weight Loss Using Mouse Diet-Induced Obesity Test

The experiments were performed using a diet-induced obesity (DIO) test. The DIO test is a widely accepted animal model of human obesity well mimicking the development and course of the disease, its comorbidities as well as its response to pharmacological medications (Hariri and Thibault Nutr. Res. Rev. 23:270-299 2010). Young (22-25 g) male C57B16 mice were fed with high-fat diet (e.g. D12492, Research Diets Inc., New Brunswick, N.J., USA), thus these animals became obese compared to mice kept on a control diet. The body weights of control and obese mice were 31.84±0.89 and 45.74±0.78 g, respectively, in the first experiment (n=8 per group, mean±SEM). These values were 30.47±0.58 and 45.38±0.61 g (n=20 per group) in the second experiment, and 27.61±0.34 and 41.00±0.91 (n=8 per group) in the third experiment (Study 2). Then the animals were assigned to different groups and treated with different vehicles or test compounds. Test compounds were dissolved in water and administered per os twice daily in a volume of 10 ml/kg of body weight. The animals were weighed daily during the experiments. The percent weight loss of each animal compared the baseline body weight was calculated at the end of the two-week-long experiment. The average percent weight change of the vehicle group was subtracted from the percent weight change of each animal, thus the vehicle-corrected weight loss was calculated. The statistical evaluation of results included descriptive statistics (mean and standard error). Furthermore, statistical significance of differences between groups was evaluated using one-way or factorial ANOVA test followed by Duncan's post-hoc test. Interactions between different treatments (i.e. combination of drugs) were evaluated using factorial ANOVA test.

### Study 1

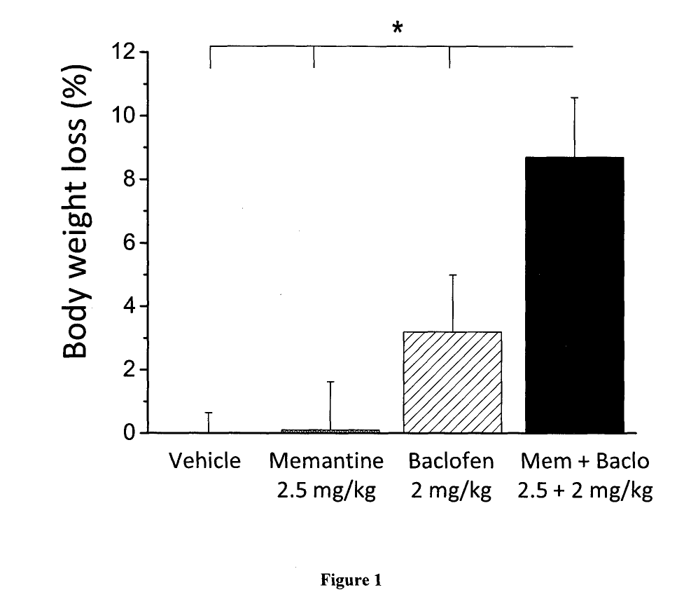

Studying the effects of 2.5 mg/kg memantine and 2 mg/kg baclofen separately and in combination, we found that memantine did not influence the body weight of treated animals compared to vehicle controls (body weight loss: 0.1%). Baclofen caused a body weight loss of 3.2%, which was statistically non-significant. In contrast, treatment with the combination of memantine and baclofen caused 8.7% decrease in the body weight. The body weight loss of the group treated with the combination was statistically significantly higher (p<0.05, one-way ANOVA and Duncan's post hoc test) than in the vehicle, memantine alone or baclofen alone groups (FIG. 1). The doses shown in FIG. 1 were administered per os twice daily. The results are shown as mean±SE of percent vehicle-corrected weight loss. There were no statistical differences between the other three groups. (Number of animals: n=8 per group, except the memantine group, where n=7.)

These data suggested a striking synergistic effect. Therefore, we repeated the experiment with larger group sizes (n=20 per group) in order to statistically assess the likelihood of this apparent synergy.

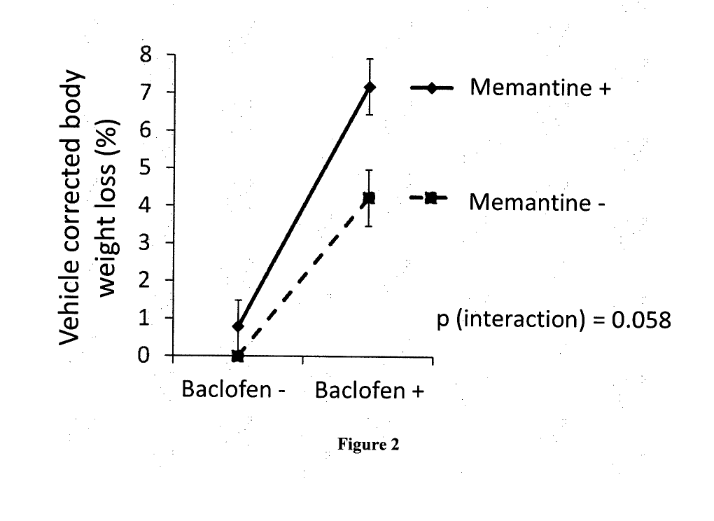

Synergy is considered to be statistically proven if the factorial ANOVA test with the two drug treatments as two factors indicates a significant interaction between the two factors (Slinker J. Mol. Cell. Cardiol. 30:723-731 1998). The null hypothesis of the interaction is that the two drugs exert their effect independently from each other, therefore these effects are summed up when the drugs are administered in combination (linear additivity). Rejection of the null hypothesis (significant interaction) means a significant difference between the sum of the effect of the two drugs (administered alone) and the effect of the combination (contra- or supraadditivity, depending on the direction of deviation). If the data were obtained from more than one experiment, then the experimental tier makes a third factor in the statistical analysis. Therefore, the results of the first (small group size, n=8) and second (n=20) experiment were pooled in the final analysis and a factorial ANOVA was performed on these data with the following three factors: 1: memantine treatment; 2: baclofen treatment; 3: experimental tier. Pooling of the datasets from the two experiments is justified because the experimental tier as factor did not produce significant alteration either alone (p=0.648), or in interaction with any of the treatments (memantine×experiment: p=0.429; baclofen×experiment: p=0.648). In contrast, both memantine and baclofen treatments as factors showed a highly significant effect (memantine: p=0.010; baclofen: p<0.001). The probability of the absence of interaction between memantine and baclofen treatments was p=0.058. This result confirms a strong, at least additive interplay between the effects of the two drugs, and also implies with a high probability (94.2%) the potential presence of a supra-additive interplay (synergy) (FIG. 2). The data in FIG. 2 are presented as mean±SE of percent vehicle-corrected weight loss. The statistical analysis of the synergistic interaction was performed using factorial ANOVA (Slinker J. Mol. Cell. Cardiol. 30:723-731 1998). Group sizes: n=28 per group (except the memantine group, where n=27), In this kind of graph (FIG. 2), parallel lines would represent additivity, rightward divergent lines indicate synergistic interaction.

These experimental results indicate an apparent interaction whereby memantine alone does not exert a considerable weight-reducing effect but potentiates the weight reducing effect of baclofen. This interpretation was supported by the finding that weight loss of memantine-treated group did not differ significantly from the weight loss of vehicle-treated group either in the first experiment, or in the pooled dataset (first experiment: p=0.957, pooled dataset: p=0.449, Duncan's post hoc test). In contrast, when memantine was administered in combination with baclofen, it increased the effect of baclofen significantly, as the weight loss of the group receiving the combination was significantly higher than the weight loss of the group receiving baclofen alone (first experiment: p=0.019, pooled dataset: p=0.005, Duncan's post hoc test).

### Study 2

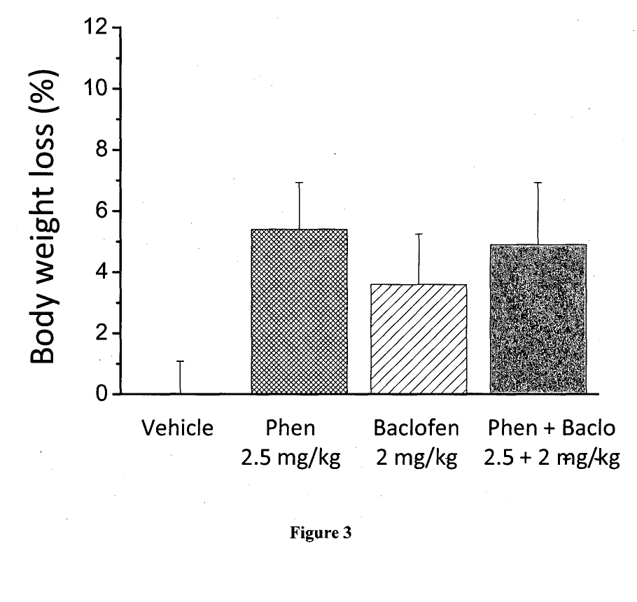

We have also tested the combination of phentermine and baclofen. In the study investigating the effects of 2.5 mg/kg phentermine and 2 mg/kg baclofen, both drugs decreased the body weight moderately (by 5.4% and 3.6%, respectively). However, the body weight loss of animals receiving the combination of these drugs (4.9%) did not exceed the body weight loss caused by phentermine alone. There was no significant difference between the groups (including the vehicle group as well). Although the group sizes (n=8 per group) were relatively small, these results clearly showed an infra-additive interaction between the two drugs when applied in combination (FIG. 3). Data in FIG. 3 are presented as mean±SE of percent vehicle-corrected weight loss.

In conclusion, studying the efficacy of combined treatments on weight loss showed that there is a synergistic (supra-additive) or at least additive interaction between memantine and baclofen in terms of their weight-reducing effect. On the other hand, it has also been shown that combining two drugs having moderate weight-reducing effects does not necessarily result in additive or synergistic interaction.

### Studies on Side Effects in Mice

### Examination of Rotarod Performance Impairing Effects in Mice

The rotarod test is a widely used simple and objective method to detect the side effects affecting motor function in rodents. This method is also capable for sensitive detection of a central muscle relaxant effect, which is a pharmacological feature of both memantine and baclofen (Farkas et al. J. Pharmacol. Toxicol. Methods 52:264-273 2005). However, other CNS side effects (e.g. sleepiness, disturbances in coordination) also impair rotarod performance. Therefore this method was used to assess liability of the combination memantine and baclofen to produce unwanted side effects.

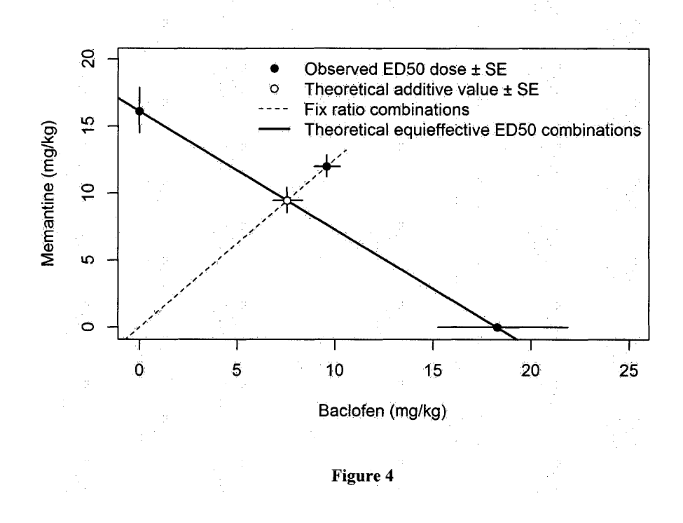

Mice were placed on a rod rotating with a constant speed of 12 rpm. After training, i.e. after habituation to the rotarod three times for 120 seconds on the previous day, the mice are usually able to run on the rod for 120 seconds without falling down. Immediately before the treatment with the test compounds, the mice were tested and only those able to stay on the rod for 120 seconds were involved to the experiment. Test compounds were dissolved in distilled water (vehicle) and administered orally to groups of ten male NMRI mice (20-24 g). One group was treated with vehicle in each experimental session. Dose-response relationships for memantine, baclofen and their combination were investigated in three separate experimental sessions. We calculated the mean latency to fall in each group and the percent change relative to the latency in the vehicle group. ED₅₀ values (effective doses causing 50% failure rate) were calculated for each compound and treatment type using logistic regression in order to characterize the effect of the compounds. The statistical analysis of the latency to fall was performed using ANOVA test followed by Duncan's post hoc test. In order to reveal whether the effect of the combination is higher than the effect of the compounds alone and also to clarify whether a supra-additive or infra-additive interaction is present, we performed an isobolographic analysis (Tallarida et al. Psychopharmacology (Berlin) 133:378-382 1997). The results are presented in Table 1 and FIG. 4.

TABLE 1
The time to falling (mean and SE) in the rotarod test

| Memantine [mg/kg] | 0 | 5 | 10 | 20 | |
|---|---|---|---|---|---|
| Mean latency to fall (sec) | 120 | 112.7 | 96.3 | 41.8** | |
| SE | 0.0 | 7.3 | 12.5 | 10.1 | |
| Baclofen [mg/kg] | 0 | 5 | 10 | 20 | |
| Mean latency to fall (sec) | 118.1 | 106.7 | 94.2 | 53.7** | |
| SE | 2 | 9 | 12.4 | 13.5 | |
| Memantin [mg/kg] + | 0 | 2.5 | 5 | 10 | 20 |
| Baclofen [mg/kg] | 0 | 2 | 4 | 8 | 16 |
| Mean latency to fall (sec) | 120 | 120 | 106.9 | 85.8** | 7.6** |
| SE | 0 | 0 | 9.1 | 12.7 | 0.9 |

All latency values presented in Table 1 show means (and SE—standard error of mean) from 10 animals. The dose “0” stands for the group receiving only vehicle (distilled water). Measurements were performed 60 minutes after treatment. **: p<0.01 (ANOVA followed by Dunnett's post hoc test; no asterisks: p>0.05).

Memantine at a dose of 20 mg/kg significantly decreased the latency to fall compared to the vehicle group. However, the doses of 5 and 10 mg/kg did not significantly decrease the latency to fall. The ED₅₀ for memantine was 16.1±1.7 mg/kg (mean±SE). Baclofen caused a significant effect also only at the dose of 20 mg/kg and its ED₅₀ (18.3±3.3 mg/kg) was comparable to that of memantine. In the case of groups treated with one of the four 1.25:1 fixed dose-ratio (1.25:1) combinations of memantine and baclofen, the two highest doses (memantine and baclofen: 10 and 8; 20 and 16 mg/kg, respectively) caused significant effect.

The calculated ED₅₀ of the combination was 21.6±1.5 mg/kg (in terms of summed equieffective doses), which was significantly higher than the theoretical ED₅₀ (17.0±1.7 mg/kg) calculated assuming additivity (FIG. 4).

It was concluded that the dose of baclofen that provided an efficient weight reducing effect in the mouse DIO test in combination with memantine (2 mg/kg/treatment) is below the dose that is eliciting muscle relaxation or other side-effects. Moreover, the dose combination showing synergy or additivity in terms of body weight reducing effects does not show synergy but an infra-additive interaction in terms of motor side effects.

### Studying the Effect on Spontaneous Motor Activity of Mice

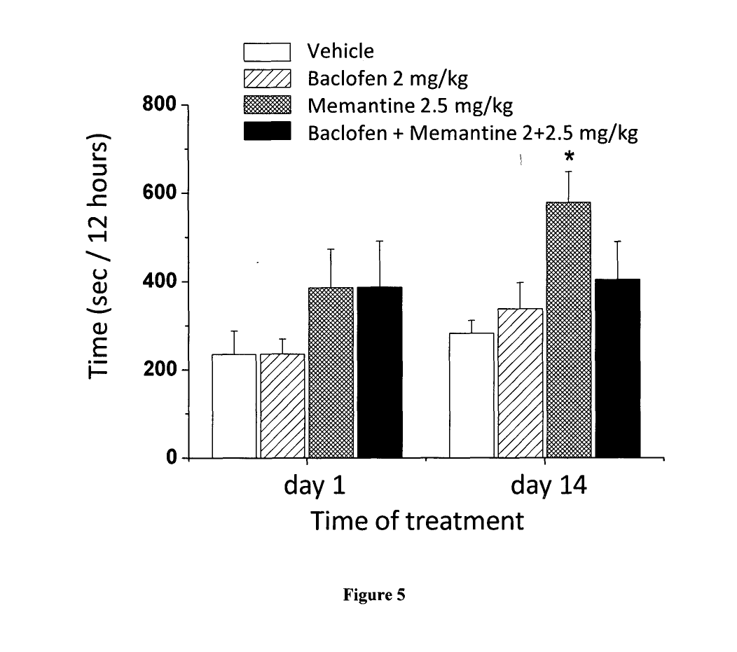

NMDA receptor antagonists are known to cause dose-dependent behavioral activation in rodents, which is manifested in increased locomotor activity (Sukhanov et al. Behav. Pharmacol. 15:263-271 2004). This behavior may correspond to side effects observed in the clinical practice, such as agitation and restlessness, which are rarely seen with memantine. We have studied the modulating effect of baclofen on the locomotor activity increasing effect of memantine when administered to mice in combination. Groups of male C57B16J mice (25-32 g; Wobe-Harlan, Hungary; 8-10 mice/group) were treated orally with 2 mg/kg baclofen, 2.5 mg/kg memantine or their combination or vehicle (distilled water) twice daily during the light phase of diurnal light-dark cycle. After one day of habituation, the activity of the animals was recorded continuously for 24 hours on the first and 14^{th} day of treatment using an automated behavioral activity measurement system (LABORAS, Metris, Netherlands). However, only the data from the light phase (12 hours) are shown. The animals were housed individually during the whole experiment in their home cages, which enabled the activity recording as well. The mechanical vibrations and gravity related static signals evoked by the movement of the animals were transformed to an electrical signal by the system, and these recorded signals were evaluated off-line by a computer algorithm (Quinn et al. J. Neurosci. Methods 130:83-92 2003). The behavior of the mice was categorized by the software as locomotor activity, immobility, climbing and grooming. FIG. 5 shows the mean and SEM of time spent with horizontal motor activity during the 12-hour light phase. Statistical analysis was performed using ANOVA followed by Tukey's post hoc test. The statistical significance of effects of drug treatments was calculated compared to vehicle and also compared to other drug-treated groups.

Studying the effects of 2.5 mg/kg memantine and 2 mg/kg baclofen alone and in combination, memantine statistically significantly (p<0.05) increased the horizontal motor activity on the 14^{th} day of treatment (an effect that is typical for NMDA antagonists). This effect of memantine did not reach statistical significance on the first day. In contrast, neither baclofen alone, nor the combination altered the motor activity either on the first or on the 14^{th} day of treatment (FIG. 5).

In conclusion, the dose-combination showing a synergistic effect in terms of weight reduction does not show a synergistic effect in terms of side effects related to spontaneous motor activity. On the contrary, baclofen apparently attenuated the locomotor activity increasing effect of memantine.

1. Combination of memantine and baclofen active ingredients.

2. The combination of claim 1 for the treatment of overweight and obesity.

3. The combination according to claim 1 or claim 2, wherein the therapeutically effective amount of memantine is in the range of about 2 to about 40 mg/day and the therapeutically effective amount of baclofen is in the range of about 5 to about 160 mg/day.

4. The combination according to any of claims 1-3, wherein the therapeutically effective amount of memantine is in the range of about 10 to about 40 mg/day and the therapeutically effective amount of baclofen is in the range of about 20 to about 160 mg/day.

5. The combination according to any of claims 1-3, wherein the therapeutically effective amount of memantine is in the range of about 2 to about 20 mg/day and the therapeutically effective amount of baclofen is in the range of about 5 to about 40 mg/day.

6. A pharmaceutical composition comprising the combination of claim 1 and pharmaceutically acceptable excipients.

7. The composition of claim 6 for use in the treatment of overweight and obesity.

8. The composition according to claim 6 or claim 7, which contains about 1 to 40 mg of memantine and about 2 to 160 mg of baclofen in each dosage unit.

9. The composition according to any of claims 6-8, wherein the composition contains about 5 to 40 mg of memantine and about 10 to 160 mg of baclofen in each dosage unit.

10. The composition according to any of claims 6-8, wherein the composition contains about 1 to 20 mg of memantine and about 2 to 40 mg of baclofen in each dosage unit.

11. Method of treatment of overweight and obesity in a mammal, particularly in human characterized by administering the therapeutically effective amount of memantine and baclofen in combination simultaneously or subsequently to the mammal to be treated.

12. The method according to claim 11, wherein the therapeutically effective amount of memantine is in the range of about 2 to about 40 mg/day and the therapeutically effective amount of baclofen is in the range of about 5 to about 160 mg/day.

13. The method according to claim 11 or claim 12, wherein the therapeutically effective amount of memantine is in the range of about 10 to about 40 mg/day and the therapeutically effective amount of baclofen is in the range of about 20 to about 160 mg/day.

14. The method according to claim 11 or claim 12, wherein the therapeutically effective amount of memantine is in the range of about 2 to about 20 mg/day and the therapeutically effective amount of baclofen is in the range of about 5 to about 40 mg/day.

15. Method of treatment according to claims 11-14 characterized by administering the pharmaceutical composition according to claim 6 to the subject to be treated.

16. The method according to claim 15, wherein the pharmaceutical composition contains memantine in the range of about 1 to about 40 mg in each dosage unit and baclofen in the range of about 2 to about 160 mg in each dosage unit.

17. The method according to claim 15 or claim 16, wherein the pharmaceutical composition contains memantine in the range of about 5 to about 40 mg in each dosage unit and baclofen in the range of about 10 to about 160 mg in each dosage unit.

18. The method according to claim 15 or claim 16, wherein the pharmaceutical composition contains memantine in the range of about 1 to about 20 mg in each dosage unit and baclofen in the range of about 2 to about 40 mg in each dosage unit.
